(19) United States
(12) Patent Application Publication
(10) Pub. No.: US 2016/0157844 A1
(43) Pub. Date: Jun. 9, 2016

(54) SOCK ANCHOR AND METHOD

(71) Applicant: Smith & Nephew, Inc., Memphis, TN (US)

(72) Inventor: Mark R. Guy, York (GB)

(21) Appl. No.: 14/559,575

(22) Filed: Dec. 3, 2014

(51) Int. Cl. A61B 17/04

(52) U.S. Cl. CPC A61B 17/0401; A61B 2017/0409

(57) ABSTRACT

Embodiments of the invention include devices and methods for providing an anchor in tissue. An anchor of some embodiments is formed from strands of material combined together into a body and one or more elongated members extending from a distal end of the body that include one or more of the strands of material from which the proximal end of the body is formed. The elongated members may be pulled back through the body and pulled proximally relative to the body to laterally expand the body. Some embodiments may also use an inserter with a rod sized to fit in a cannula through the body of the anchor and a tube configured to provide a more rigid structure about the exterior of the anchor during insertion.

### FIELD OF THE INVENTION

The present invention relates generally to the field of tissue anchors, and more particularly relates to suture anchors or tissue anchors along with their implantation instruments and related methods. Some embodiments include an anchor made from strands of material formed into a body, and some or all of the strands may be at least in part further formed into elongated members. The elongated members may be passed back through the body of the anchor. The strands may further be pulled proximally relative to the body to cause the body to expand laterally and be secured in tissue such as bone.

### BACKGROUND

it is a continuing challenge to provide devices for anchoring in tissue such as bone that are reliably strong, convenient and effective to deploy, and cost-effective. One useful type of anchor known in the prior art is an all-suture anchor. Common all-suture anchors thread a suture or tape through another smaller piece of suture or tape. The threading may include multiple penetrations or weaving through the smaller piece of suture or tape. These all-suture anchors may be deployed by pushing the combined suture or tape constructs into tissue and then pulling on free ends of the suture or tape to bunch together the constructs in the tissue and create an anchoring for the suture or tape. Prior art anchors have had varying degrees of success. Some types have provided inconsistent pull-out strength capacities. Some have required a relatively large footprint because of the bulk of anchoring portions of the devices. Some have not included configurations and instrumentation that enable the constructs to be efficiently and consistently deployable in tissue.

It would be advantageous to provide anchors, anchor systems, and methods that enable predictable and consistently strong anchoring in tissue. It would also be advantageous to provide for convenient and effective deployment of anchors enable by the design of the anchors and by the instruments and methods used to implant the anchors.

### SUMMARY

An embodiment of the invention is an anchor or anchor system with an anchor formed from strands of material combined together into a body having a proximal end and a distal end and one or more elongated members extending from the distal end of the body. The body may be formed from the strands of material such that a cannula is created through the body, and the one or more elongated members extending from the distal end of the body may include one or more of the strands of material from which the proximal end of the body is formed. The distal ends of the one or more elongate members in some embodiments are sized to fit in combination through the cannula in the body and extend from the proximal end of the body.

In some embodiments, the body may be a woven tubular body, the cannula through the body may extend through the entire length of the body between the proximal and distal ends of the body, the body may include reinforcing materials that are stiffer than the one or more strands and are positioned within the body so that the body deforms in a predetermined manner when the elongated members are tensioned proximally relative to the body, and the body may include a collar near the proximal end of the body that is configured to stiffen the proximal end of the body. In some embodiments, there may be more than two elongated members extending from the distal end of the body that include one or more of the strands of material from which the proximal end of the body is formed or there may be two, three, four or more elongated strands extending from the distal end of the body that include one or more of the strands of material from which the proximal end of the body is formed. In some embodiments, the one or more elongated members extending from. the distal end of the body may each be formed from multiple strands of material from which the proximal end of the body is formed. In some embodiments, the distal ends of the one or more elongated members may extend through a distal end of the cannula in the body and extend from a proximal end of the cannula, and the one or more elongated members may not penetrate the body from within the cannula between the distal end of the cannula and the proximal end of the cannula where the elongated members extend through the distal end of the cannula and from the proximal end of the cannula.

Another embodiment of the invention is an anchor system that includes an anchor. and an inserter. The anchor may be formed from strands of material combined together and include a body having a proximal end and a distal end, The body may be formed from the strands of material such that a cannula is created through the body, and one or more elongated members may extend from the distal end of the body that include one or more of the strands of material from which the proximal end of the body is formed. The inserter may include a rod sized to fit in the cannula through the body of the anchor, a wall from which the rod extends distally, the wall being configured to contact the proximal end of an anchor when the anchor is placed over the rod, and a tube configured to provide a more rigid structure about the exterior of the anchor during insertion. The tube may have an inside diameter into which the rod, the wall, and the anchor fit.

In some embodiments, the rod may be stepped from smaller to larger diameters from the rod's distal end toward the rod's proximal end, or the rod may be tapered from a smaller diameter to a larger diameter from the rod's distal end toward the rod's proximal end. In sonic embodiments, the wall may include one or more lumens through which the one or more elongated members may be passed.

Yet another embodiment of the invention is a method of anchoring suture into tissue that may include providing an anchor formed from strands of material combined together into a body having a proximal end and a distal end, wherein the body is formed from the strands of material such that a cannula is created through the body, and one or more elongated members extending from the distal end of the body that include one or more of the strands of material from which the proximal end of the body is formed. The method may further include positioning the one or more elongated members through the body by moving distal ends of the elongated members through the distal end of the body, through the cannula, and out the proximal end of the body, and inserting the anchor into tissue. Method embodiments may also include putting the elongated members proximally to cause the body to expand laterally, and employing the elongated members in a surgical procedure.

In some embodiments, the act of providing an anchor may include providing an anchor wherein the one or more elongated members do not penetrate the body from within the cannula between a distal end of the cannula and a proximal end of the cannula beyond the distal end of the body. some embodiments, the act of inserting the anchor into tissue may include inserting the anchor into bone. In some embodiments, the act of inserting the anchor into tissue may include inserting the anchor with an inserter including a rod sized to fit in the cannula through the body of the anchor to support the anchor. In some embodiments, the act of inserting the anchor into tissue may include inserting the anchor with an inserter including a tube configured to provide a more rigid structure about the exterior of the anchor during insertion, the tube having an inside diameter into which the rod and the anchor fit. In some embodiments, when the act of pulling the elongated members proximally is accomplished, an inserter may be held in contact with a proximal end of the anchor while the body is expanded laterally.

### BRIEF DESCRIPTION OF THE DRAWINGS

FIG. 1 is a perspective view of an embodiment of an anchor in the process of being woven.

FIG. 2 is a perspective view of an embodiment of an anchor with elongated members extending from a distal end of a body of the anchor.

FIG. 3 is a perspective view of an embodiment of an anchor with ribs in a portion of the anchor to encourage a particular deformation of the anchor,

FIG. 4 is a perspective view of a proximal end of an embodiment of an anchor with a collar configured to provide rigidity to the proximal end of the anchor.

FIG. 5 is a perspective view of an embodiment of an anchor into which an inserter has been placed.

FIG. 6 is a distal end elevation view of an embodiment of an inserter.

FIG. 7 is a perspective view of a distal end of an embodiment of an inserter with some components illustrated as transparent.

FIG. 8 is a perspective view of a distal end of an embodiment of an inserter with some components illustrated as transparent.

FIG. 9 is a perspective view of a distal end of an embodiment of an inserter with some components illustrated as transparent.

FIGS. 10A-10E are perspective views illustrating a sequence of preparing and expanding an embodiment of an anchor.

### DETAILED DESCRIPTION

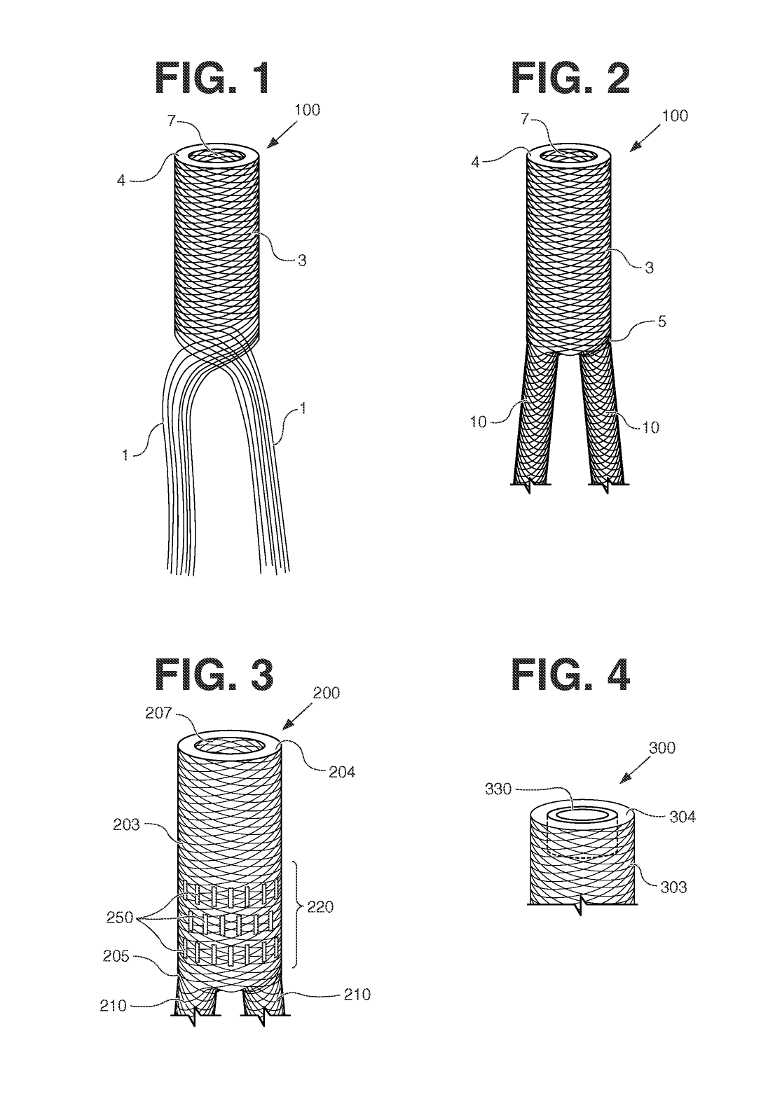

An embodiment of an anchor 100 is illustrated in FIGS. 1 and 2 and is formed from strands 1 of material. The strands 1 shown are combined together into a body 3 and into two elongated members 10 (FIG. 2). The overall strength of the anchor 100 may be enhanced due to the strands 1 continuing between and being part of both the body 3 and elongated elements 10. The body 3 depicted includes a proximal end 4 and a distal end 5. The body 3 may be woven, braided, or otherwise formed from the strands 1 such that a cannula 7 is created through the body 3. As shown in FIGS. 1 and 2, the cannula 7 provides a substantially round opening through the body 3 that extends through the entire length of the body 3 between the proximal end 4 and the distal end 5 of the body 3. However, in other embodiments, a cannula may be any other functional opening in a body and does not need to be substantially round, be continuous around its periphery, extend through the entire length of the body, or be symmetrical. The body 3 of the illustrated embodiment may also be described as a tubular body.

An embodiment of an anchor 200 is illustrated in FIG. 3 with a body 203 formed from one or more strands. The body 203 has a proximal end 204 and a distal end 205. The anchor 200 is similar to the anchor 100 but includes a deformation zone 220. The deformation zone 220 is configured to collapse in a predetermined way when subjected to compression between the proximal end 204 and distal end 205 of the body 203. Such compression may be generated by pulling distal ends of elongated members 210 back through a cannula 207 in the body 203. The body 203 includes reinforcing materials 250 that are stiffer than the one or more strands and are positioned within the body 203 so that the body deforms in a predetermined manner when the elongated members 210 are tensioned relative to the body 203, where the elongated members 210 have been pulled back through the cannula 207 prior to tensioning. The reinforcing materials 250 shown in this embodiment are placed in rows relative to a longitudinal axis of the body 203 about all or substantially all of the radius of the body 203. Therefore, when the body 203 is compressed by tensioning the elongated members 210 that have been pulled back through the cannula 207, the reinforced portions (reinforced with the reinforcing materials 250) of the body 203 are less prone to buckle, and the body tends to buckle first at the regions that are not reinforced with the reinforcing materials 250. This action results in lateral expansion of the body 203, which contributes to fixing of the body 203 within tissue into which it has been fit in an unexpanded state. Any other configuration of one or more reinforcing materials that leads to buckling, bunching, gathering, accordion type behavior, or any other lateral expansion in response to forces applied may be used in other embodiments. Reinforcing materials may be used to induce deformations in other embodiments of an anchor, such as but not limited to the anchor 100.

A proximal end 304 of a body 303 of an anchor 300 is illustrated in FIG. 4. The body 303 includes a collar 330 near the proximal end 304 that is configured to stiffen the proximal end 304 of the body 303. The term “stiffen” as used herein means to increase rigidity relative to strands or combined strands from which the body 303 is formed. In some embodiments, stiffening is provided to resist turning the entire body 303 inside-out when elongated members, such as the elongated members 10 and 210 described in association with FIGS. 2 and 3 are passed through a cannula in a body and tensioned to compress the body, The collar 330 may also provide for a stiffened abutment of the proximal end 304 of the body 303 against an inserter. The collar 330 may be substantially round, as illustrated in FIG. 4, or may be of any effective shape or size to stiffen a portion of a body into which it is incorporated. The collar 303 illustrated extends within the entire periphery of the proximal end 304 of the body 303. The term “collar” as used herein is not limiting regarding shape or size of elements within the invention that may be used to stiffen a body. Any other embodiment of an anchor, for example and without limitation, the anchor 100 or the anchor 200, may include a collar configured to stiffen a proximal end of their respective bodies.

Two elongated members 10 are shown in FIG. 2, but other embodiments may include only one elongated member or more than two elongated members. The elongated members 10 extend from the distal end 5 of the body 3 and include multiple strands 1 of material from which the proximal end 4 of the body 3 is formed. In the illustrated embodiment, the body 3 is formed by weaving. The elongated member or members of some embodiments may include only one strand 1 or may include more than two strands 1. The elongated members of some embodiments that include multiple elongated members may include the same number or different numbers of strands in each elongated member. The two elongated members 10 depicted are substantially radially equidistantly spaced about a longitudinal axis of the body 3. Other embodiments may include elongated members that are not radially equidistantly spaced. Such a non-radially equidistant spacing may promote bunching of a body to which the elongated members are coupled because of uneven forces being applied relative to the longitudinal axis of the body when the elongated members are pulled back through the body. The elongated members 210 of the anchor 200 and elongated members (not shown) of the anchor 300 are essentially similar to the elongated members 10 described in association with the anchor 100.

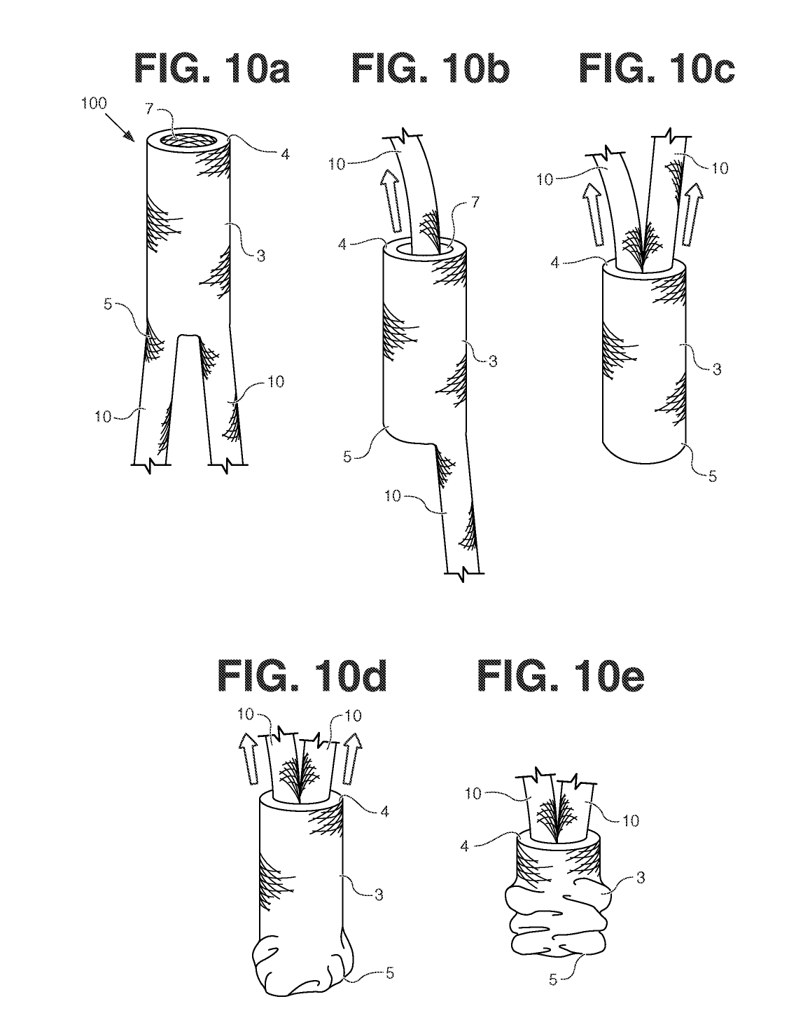

Distal ends of the elongated members 10 are sized to fit in combination through the cannula 7 in the body 3 and extend from the proximal end 4 of the body 3. As illustrated in FIGS. 10C-10E, the elongated members 10 extend both through a distal end of the cannula 7 in the body 3 and extend from a proximal end of the cannula 7. In other embodiments, one or more elongated members may penetrate a body from within a cannula in the body, may penetrate the body from outside of the body, and may exit the cannula from the proximal end of the cannula. However, in the illustrated embodiment, the elongated members 10 do not penetrate the body 3 other than through the distal and proximal ends of the cannula. In other words, the elongated members 10 do not penetrate the walls of the cannula body. Specifically in the embodiment depicted, the elongated members 10 do not penetrate the body 3 from within the cannula 7 between the distal end of the cannula 7 and the proximal end of the cannula 7, and the elongated members 10 extend through the distal end and the cannula 7 and from the proximal end of the cannula 7.

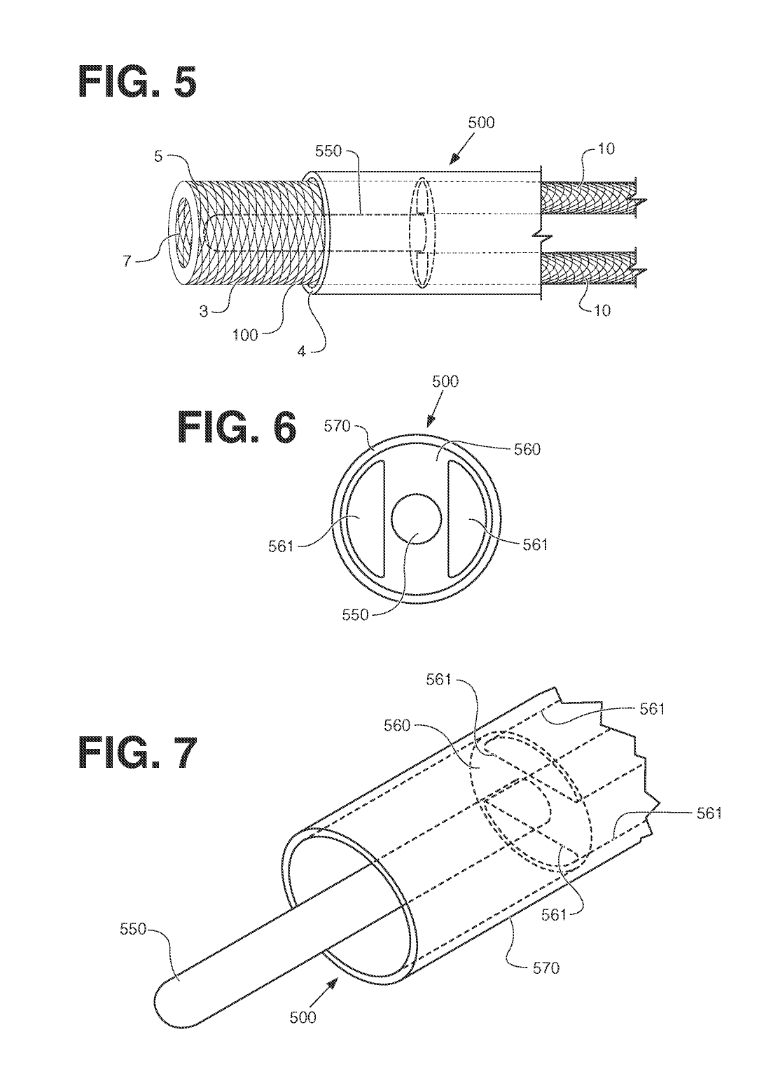

An embodiment of the invention is an anchor system that includes an anchor, such as the anchor 100, and an inserter, such as the inserter 500 shown in FIGS. 5-7. An anchor of the anchor system may also be one of the anchors 200, 300 or may be any other anchor capable of functioning with the inserter 500 and other similar inserters. The inserter 500 shown in FIGS. 5-7 includes a rod 550 sized to fit through, or at least partially within, the body 3. In particular, the rod 550 is sized to fit in the cannula 7 through the body 3 of the anchor 100. The rod 550 illustrated has a continuous diameter along its longitudinal axis. However, in other embodiments, a rod may have a discontinuous diameter along its longitudinal axis to enhance its ability to interact with an anchor or for other reasons.

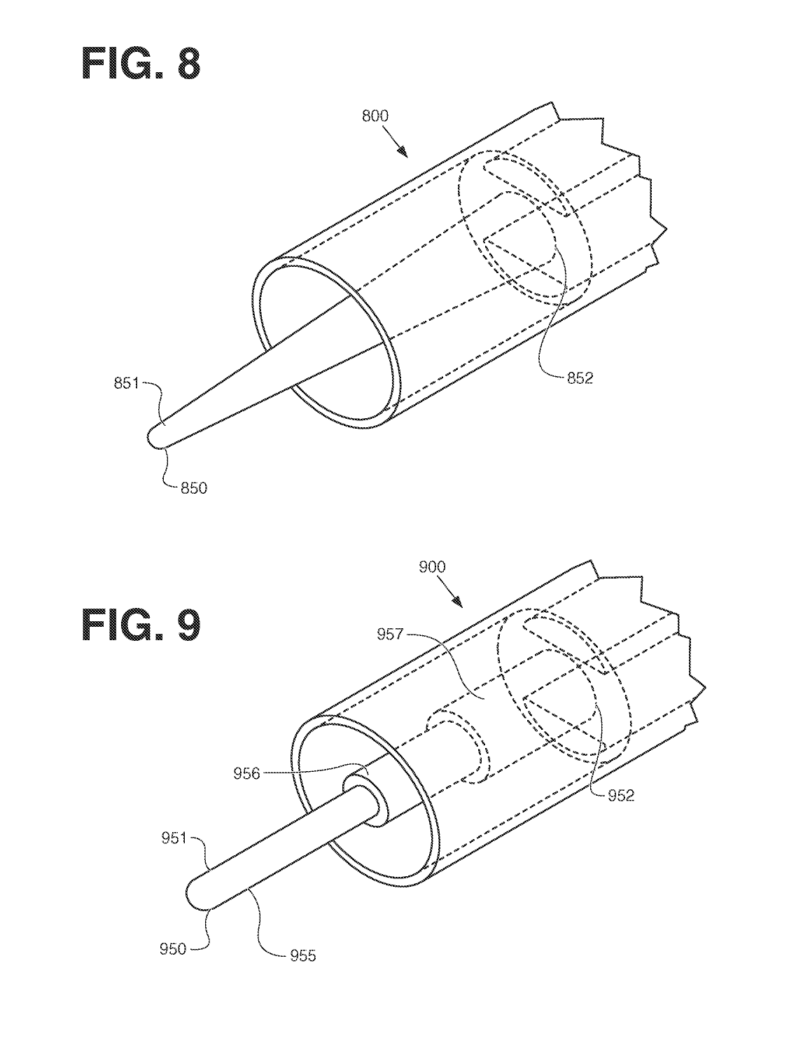

Two examples of rods with discontinuous diameters are illustrated in FIGS. 8 and 9. A rod 850 is illustrated in FIG. 8 as part of an inserter 800. The rod 850 is tapered from a smaller diameter at its distal end 851 to a larger diameter at its proximal end 852. The taper shown is a linear taper where the diameter reduction is substantially constant from the distal end 851 to the proximal end 852. The rate of change in taper may be non-linear in other embodiments. This configuration may be useful in providing a tighter fit between an anchor and the rod 850 proximally where the anchor is intended to remain rigid during deployment and in providing a looser fit and a more easily guided coupling and deployment at the distal end 851 adjacent to the deformation zone 220 (FIG. 3, for example).

A rod 950 is illustrated in FIG. 9 as part of an inserter 900. The rod 950 includes diameters that are stepped from smaller to larger from a distal end 951 to a proximal end 952 of the rod 950. Various embodiments may include any number of steps as may be effective in assisting with deployment of an anchor. The rod 950 depicted includes three stepped diameter sections 955, 956, 957. Similar to the rod 850, the configuration of the rod 950 may be useful in providing a tighter fit between an anchor and the rod 950 proximally where the anchor is intended to remain rigid during deployment and in providing a looser fit and a more easily guided coupling and deployment at the distal end 951 adjacent to the deformation zone 220.

Returning to FIGS. 6 and 7, the inserter 500 is shown including a wall 560 from which the rod 550 extends distally. The wall 560 illustrated is configured to contact the proximal end 4, 204, 304 of the anchor 100, 200, 300 when the anchor 100, 200, 300 is placed over the rod 550. The wall 560 shown is substantially as large as the proximal end 4, 204, 304 of the anchor 100, 200, 300. However, in other embodiments, a wall may be only a fraction of the size of a proximal end of an anchor and provide a stop against which an anchor may be supported. The wall 560 includes two lumens 561 through which elongated members such as the elongated members 10, 210 may be passed. Walls of various embodiments may include a single lumen or more than two lumens through which one or more elongated member may be passed.

The inserter 500 shown in FIGS. 6 and 7 includes a tube 570 configured to provide a more rigid structure about the exterior of an anchor while the anchor is being inserted. The tube 570 has an inside diameter into which the rod 550, the wall 560, and an anchor such as the anchor 100 will fit simultaneously. The inserters 800, 900 shown in FIGS. 8 and 9 include similar walls and tubes as described in association with the inserter 500.

An embodiment of the invention is a method of anchoring suture into tissue. Several acts of a method are illustrated in a viable sequence in FIGS. 10A-10E. An inserter 500, 800, 900 that may be used to perform an insertion step has been omitted from FIGS. 10A-10E for clarity in showing the actions and responses of the anchor 100. Method embodiments may include providing an anchor formed from strands of material combined together. An anchor provided, such as the anchor 100, may include a body 3 having a proximal end 4 and a distal end 5 (FIGS. 1, 2, and 10A-10E). The body 3 may be woven from the strands 1 of material such that a cannula 7 is created through the body 3. The provided anchor 100 may also include one or more elongated members 10 extending from the distal end 5 of the body 3 that include one or more of the strands 1 of material from which the proximal end 4 of the body is formed. All of the variations and alternative embodiments of the anchors described herein, including but not limited to anchors 100, 200, 300, may be included in anchors provided to accomplish method embodiments further described herein.

An act of some method embodiments includes positioning one or more elongated members through the body by moving distal ends of the elongated members through the distal end of the body, through the cannula, and out the proximal end of the body. An example of these actions is illustrated in FIGS. 10A-10C. In FIG. 10B, a first elongated member 10 is shown being positioned through the body 3 by moving a distal end of the elongated member 10 through the distal end 5 of the body 3, through the cannula 7, and out of the proximal end 4 of the body 3. In FIG. 10B, a second elongated member 10 is shown being positioned through the body 3 by moving a distal end of the elongated member 10 through the distal end 5 of the body 3, through the cannula 7, and out of the proximal end 4 of the body 3. At this stage, an inserter such as the inserter 500 may be coupled with the anchor 100, as partially illustrated in FIG. 5. The inserter 500 includes the rod 550 sized to fit in the cannula 7 through the body 3. The rod 550 supports the anchor 100 during insertion. Inserters of some embodiments may also include features such as the wall 560 and the tube 570 that may further aid with insertion of the anchor 100, as described herein. Prior to seating of the wall 560 against the proximal end 4 of the anchor 100, the elongated members 10 may be passed through one or more of the lumens 561 and fed through a proximal end of the inserter 500 where a user may tension or release the elongated members when performing a surgical procedure.

Another act of some method embodiments includes pre-drilling one or more holes in tissue, such as but not limited to bone, prior to inserting an anchor into the tissue. In other embodiments, an inserter may include a self-drilling or other penetrating feature to provide an opening in tissue into which an anchor may be deployed. In the state illustrated in FIG. 10C, the anchor 100 may be inserted into tissue. After insertion into tissue an act of some method embodiments may include pulling or tensioning the elongated members 10 proximally relative to the body 3 to cause the body 3 to expand laterally, as shown in FIGS. 10D and 10E. A partial deployment is illustrated in FIG. 10D. After further tensioning of the elongated members 10 proximally relative to the body 3, the anchor 100 is fully deployed laterally, as illustrated in FIG. 10E. When used in combination with the inserter 500 and other similar devices, the act of pulling the elongated members 10 proximally may be accomplished while the inserter 500 or another similar device is held in contact with the proximal end 4 of the anchor 100 while the body 3 is expanded laterally. Method embodiments may further include employing one or more elongated member that are anchored in tissue in a surgical procedure. Any surgical procedures that may be accomplished with elongated members, such as but not limited to sutures or surgical tape, may be accomplished with the anchor system when deployed. The anchor system may further be used in combination with other surgical equipment.

Various embodiments of a system wholly or its components individually may be made from any biocompatible material that has physical properties conducive to its use in such a system or one or more of its components. For example and without limitation, biocompatible materials may include in whole or in part: non-reinforced polymers, reinforced polymers, metals, ceramics, adhesives, reinforced adhesives, and combinations of these materials. Reinforcing of polymers may be accomplished with carbon, metal, or glass or any other effective material. Examples of biocompatible polymer materials include polyamide base resins, polyethylene, Ultra High Molecular Weight (UHMW) polyethylene, low density polyethylene, polymethlmethacrylate (PMMA), polyetheretherketone (PEEK), polyetherketoneketone (PEKK), a polymeric hydroxethylmethacrylate (PHEMA), and polyurethane, any of which may be reinforced. Example biocompatible metals include stainless steel and other steel alloys, cobalt chrome alloys, zirconium, oxidized zirconium, tantalum, titanium, titanium alloys, titanium-nickel alloys such as Nitinol and other superelastic or shape-memory metal alloys. Strands or elongated members of the anchors or other components of the invention may be single strand, woven, braided, or any combination thereof from any of these or other biocompatible materials. The strands or elongated members may be any effective natural or synthetic material and may be a use or combination of materials well-known in the art, Strands or elongated members of various embodiments may be resorbable or not resorbable.

Terms such as proximal, lateral, adjacent, over, and the like have been used relatively herein. However, such terms are not limited to specific coordinate orientations, distances, or sizes, but are used to describe relative positions referencing particular embodiments. Such terms are not generally limiting to the scope of the claims made herein. Any embodiment or feature of any section, portion, or any other component shown or particularly described in relation to various embodiments of similar sections, portions, or components herein may be interchangeably applied to any other similar embodiment or feature shown or described herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. An anchor formed from strands of material combined together comprising:
a body having a proximal end and a distal end, wherein the body is formed from the strands of material such that a cannula is created through the body; and
one or more elongated members extending from the distal end of the body that include one or more of the strands of material from which the proximal end of the body is formed;
wherein distal ends of the one or more elongate members are sized to fit in combination through the cannula in the body and extend from the proximal end of the body.

2. The anchor of claim 1 wherein the body is a woven tubular body.

3. The anchor of claim 1 wherein the cannula through the body extends through the entire length of the body between the proximal and distal ends of the body.

4. The anchor of claim I wherein the body includes reinforcing materials that are stiffer than the one or more strands and are positioned within the body so that the body deforms in a predetermined manner when the elongated members are tensioned proximally relative to the body.

5. The anchor of claim I wherein the body includes a collar near the proximal end of the body that is configured to stiffen the proximal end of the body.

6. The anchor of claim I wherein there are more than two elongated members extending from the distal end of the body that include one or more of the strands of material from which the proximal end of the body is formed.

7. The anchor of claim 1 wherein the one or more elongated members extending from the distal end of the body are each formed from multiple strands of material from which the proximal end of the body is formed.

8. The anchor of claim I wherein the distal ends of the one or more elongated members extend through a distal end of the cannula in the body and extend from a proximal end of the cannula.

9. The anchor of claim 8 wherein the one or more elongated members do not penetrate the body from within the cannula between the distal end of the cannula and the proximal end of the cannula where the elongated members extend through the distal end of the cannula and from the proximal end of the cannula.

10. An anchor system comprising:
an anchor formed from strands of material combined together comprising:
a body having a proximal end and a distal end, wherein the body is formed from the strands of material such that a cannula is created through the body, and
one or more elongated members extending from the distal end of the body that include one or more of the strands of material from which the proximal end of the body is formed; and
an inserter comprising:
a rod sized to fit in the cannula through the body of the anchor,
a wall from which the rod extends distally, the wall being configured to contact the proximal end of an anchor when the anchor is placed over the rod, and
a tube configured to provide a rigid structure about the exterior of the anchor during insertion, the tube having an inside diameter into which the rod, the wall, and the anchor fit.

11. The anchor system of claim 10 wherein the cannula through the body extends through the entire length of the body between the proximal and distal ends of the body.

12. The anchor system of claim 10 wherein the one or more elongated members extending from the distal end of the body are each woven from multiple strands of material from which the proximal end of the body is formed.

13. The anchor system of claim 10 wherein the distal ends of the one or more elongated members extend through a distal end of the cannula in the body and extend from a proximal end of the cannula.

14. The anchor system of claim 13 wherein the one or more elongated members do not penetrate the body from within the cannula between the distal end of the cannula and the proximal end of the cannula where the elongated members extend through the distal end of the cannula and from the proximal end of the cannula.

15. The anchor system of claim 10 wherein the rod is stepped from smaller to larger diameters from the rod's distal end toward the rod's proximal end.

16. The anchor system of claim 10 wherein the rod is tapered from a smaller diameter to a larger diameter from the rod's distal end toward the rod's proximal end.

17. The anchor system of claim 10 wherein the wall includes one or more lumens through which the one or more elongated members may be passed.

18. A method of anchoring suture into tissue comprising:
providing an anchor formed from strands of material combined together comprising:
a body having a proximal end and a distal end, wherein the body is woven. from the strands of material such that a cannula is created through the body, and
one or more elongated members extending from the distal end of the body that include one or more of the strands of material from which the proximal end of the body is formed;
positioning the one or more elongated members through the body by moving distal ends of the elongated members through the distal end of the body, through the cannula, and out the proximal end of the body;
inserting the anchor into tissue;
pulling the elongated members proximally to cause the body to expand laterally; and
employing the elongated members in a surgical procedure.

19. The method of claim 18 wherein the act of providing an anchor includes providing an anchor wherein the one or more elongated members do not penetrate the body from within the cannula between a distal, end of the cannula and a proximal end of the cannula beyond the distal end of the body.

20. The method of claim 18 wherein the act of inserting the anchor into tissue includes inserting the anchor into bone.

21. The method of claim 18 wherein the act of inserting the anchor into tissue includes inserting the anchor with an inserter including a rod sized to fit in the cannula through the body of the anchor to support the anchor.

22. The method of claim 18 wherein the act of inserting the anchor into tissue includes inserting the anchor with an inserter including a tube configured to provide a more rigid structure about the exterior of the anchor during insertion, the tube having an inside diameter into which the rod and the anchor fit.

23. The method of claim 18 wherein when the act of pulling the elongated members proximally is accomplished, an inserter is held in contact with a proximal end of the anchor while the body is expanded laterally.
